(19) United States
(12) Patent Application Publication
(10) Pub. No.: US 2025/0333789 A1
(43) Pub. Date: Oct. 30, 2025

(54) DETECTION METHOD FOR HUMAN UMBILICAL CORD MESENCHYMAL STEM CELLS (hUC-MSCs)

(71) Applicant: SCLnow Biotechnology Co., Ltd., Beijing (CN)

(72) Inventors: Cheng Li, Beijing (CN); Lei Guo, Beijing (CN)

(21) Appl. No.: 19/030,090

(22) Filed: Jan. 17, 2025

(30) Foreign Application Priority Data
Apr. 30, 2024 (CN) 2024105413199

(51) Int. Cl. C12Q 1/6881; C12N 15/10; C12Q 1/6806; C12Q 1/686; G01N 27/447

(52) U.S. Cl. CPC C12Q 1/6881; C12N 15/1096; C12Q 1/6806; C12Q 1/686; G01N 27/44726

(57) ABSTRACT

The present invention relates to stem cell detection, and particularly relates to a detection method for human umbilical cord mesenchymal stem cells (hUC-MSCs). RNA of hUC-MSCs is extracted and reversely transcribed into cDNA, PCR amplification is conducted on the cDNA obtained to obtain an amplification product, and electrophoresis detection is conducted on the amplification product. Nucleic acid electrophoresis has the characteristics of high efficiency, high sensitivity and high resolution, therefore, in the present solution, the four genes of SSEA-4, NANOG, OCT-4 and SOX2 are detected by nucleic acid electrophoresis, which can be efficiently separated and detected according to different nucleic acid molecular sizes thereof in a short time.

### REFERENCE TO AN ELECTRONIC SEQUENCE LISTING

The contents of the electronic sequence listing (INI0218USP_SQ_0526.xml; Size: 10 KB; and the date of creation: May 26, 2025) is herein incorporated by reference in its entirety. The XML file “INI0218USP_SQ_0526” includes no new matter as required by 37 CFR 1.835(a)(4). The substitute specification contains no new matter.

### FIELD OF THE INVENTION

The present invention relates to the technical field of stem cell detection, and particularly relates to a detection method for human umbilical cord mesenchymal stem cells (hUC-MSCs).

### BACKGROUND OF THE INVENTION

Mesenchymal stromal/stem cells (MSCs) are a kind of adult stem cells other than hematopoietic stem cells that exist in a variety of tissues (such as bone marrow, umbilical cord blood, umbilical cord tissue, placenta tissue and adipose tissue) and have a multi-directional differentiation potential. This kind of stem cells have a potential to differentiate into a variety of mesenchymal series cells (such as osteoblasts, chondroblasts and lipoblasts) or non-mesenchymal series cells, and have a unique cytokine secretion function.

Multipotency of MSCs means an ability to form more than one type of cells in organisms, which can induce differentiation into a variety of functional cells such as osteocytes and adipocytes in vitro, and can express genetic characteristics that regulate cell differentiation into other histiocyte types or organs (for example, the genes of SSEA-4, OCT-4, SOX-2 and NANOG are multipotent genes). MSCs have a characteristic of producing new cells by cell division, including a feature that the proportion of different stages in a cell cycle and the number of cells during cell division are changed over time. Regeneration factors secreted by hUC-MSCs need to be detected in order to control the quality requirements of MSCs.

### SUMMARY OF THE INVENTION

To solve the above problems, the present invention provides a detection method for human umbilical cord mesenchymal stem cells (hUC-MSCs).

To achieve the above purpose, the present invention provides the following technical solution:

The present invention provides a detection method for human umbilical cord mesenchymal stem cells (hUC-MSCs), comprising the following steps:
1) Extracting RNA of hUC-MSCs and reversely transcribing the RNA into cDNA;
2) Conducting PCR amplification on the cDNA obtained in step 1) to obtain an amplification product;

A nucleotide sequence of a forward primer of a primer pair used for amplification of factor SSEA-4 is shown in SEQ ID No.1, and a nucleotide sequence of a reverse primer sequence is shown in SEQ ID No.2;

A nucleotide sequence of a forward primer of a primer pair used for amplification of factor OCT-4 is shown in SEQ ID No.3, and a nucleotide sequence of a reverse primer sequence is shown in SEQ ID No.4;

A nucleotide sequence of a forward primer of a primer pair used for amplification of factor SOX-2 is shown in SEQ ID No.5, and a nucleotide sequence of a reverse primer sequence is shown in SEQ ID No.6;

A nucleotide sequence of a forward primer of a primer pair used for amplification of factor NANOG is shown in SEQ ID No.7, and a nucleotide sequence of a reverse primer sequence is shown in SEQ ID No.8;
3) Conducting electrophoresis detection on the amplification product obtained in step 2); when the amplified length of the amplification product is 121 bp, factor SSEA-4 is secreted beside hUC-MSCs;

When the amplified length of the amplification product is 137 bp, factor OCT-4 is secreted beside hUC-MSCs;

When the amplified length of the amplification product is 171 bp, factor SOX-2 is secreted beside hUC-MSCs;

When the amplified length of the amplification product is 94 bp, factor NANOG is secreted beside hUC-MSCs.

Preferably, a system for the reverse transcription in step 1) comprises: 1 μL of dNTP (10 mM), 1 μL of Oligo dT-joint primer (2.5 mM), 1 μL of template RNA, 0.5 μL of reverse transcriptase (500 U/μL), 0.5 μL of RNase inhibitor (40 U/μL), 4 μL of 5×RT-PCR buffer solution and 12 μL of double distilled water.

Preferably, an amplification procedure for the reverse transcription is: replenishing the dNTP, the Oligo dT-joint primer and the template RNA to 10 μL with the double distilled water, pre-denaturing at 65° C. for 5 min, and mixing with the reverse transcriptase, the RNase inhibitor, the 5×RT-PCR buffer solution and the double distilled water; a reaction procedure is: 30° C. for 10 min, 42° C. for 15 min-30 min, 70° C. for 15 min, and 4° C. for preservation.

Preferably, a system for the PCR amplification in step 2) comprises: 2 μL of 10×PCR buffer solution, 0.8 μL of dNTP Mixture, 0.4 μL of forward primer (20 μM), 0.4 μL of reverse primer (20 μM), 0.2 μL of Ex Taq HS, 2 μL of cDNA and double distilled water replenished to 20 μL.

Preferably, a procedure for the PCR amplification is: pre-denaturing at 94° C. for 5 min; denaturing at 94° C. for 30 s, annealing at 55° C. for 30 s, and extending at 72° C. for 30 s which are cycled for 35 times; extending at 72° C. for 10 min, and preserving at 4° C.

The present invention has the following beneficial effects:

Significance of SSEA-4 gene detection: stage-specific embryonic antigen gene is used as a marker of human undifferentiated multipotent embryonic stem cells.

Significance of NANOG gene detection: NANOG gene is used for expressing transcription factors in MSCs and regulating self-renewal of cells, but no mRNA thereof is found in differentiated fibroblasts.

Significance of OCT-4 gene detection: OCT-4 is a member of a POU transcription factor family, is expressed in MSCs to regulate transcription, and has a function of maintaining multipotency and self-renewal of stem cells. Detection of OCT-4 gene will cause differentiation of the stem cells. Physical characteristics of SOX2 gene: SOX2 is located on chromosome 3 of human, with a total length of 9503 bp and a coded mRNA length of 2520 bp.

Significance of SOX2 gene detection: SOX2 is a transcription factor gene; like OCT-4 and NANOG, SOX2 is used as a marker of undifferentiated stem cells; the expression level of SOX2 is decreased with the differentiation of the stem cells, and SOX2 is not expressed in fibroblasts.

Nucleic acid electrophoresis has the characteristics of high efficiency, high sensitivity and high resolution, therefore, in the present solution, the four genes of SSEA-4, NANOG, OCT-4 and SOX2 are detected by nucleic acid electrophoresis, which can be efficiently separated and detected according to different nucleic acid molecular sizes thereof in a short time.

### DESCRIPTION OF DRAWINGS

To more clearly describe the technical solution in the embodiments of the present invention or in the prior art, the drawings required to be used in the embodiments will be simply presented below.

FIG. 1 shows an internal control B-Actin and the four marker genes of NANOG, OCT4, SOX2 and SSEA4 with bands of different brightness.

### DETAILED DESCRIPTION OF THE INVENTION

The present invention provides a detection method for human umbilical cord mesenchymal stem cells (hUC-MSCs), comprising the following steps:
1) Extracting RNA of hUC-MSCs and reversely transcribing the RNA into cDNA;
2) Conducting PCR amplification on the cDNA obtained in step 1) to obtain an amplification product;

A nucleotide sequence of a forward primer of a primer pair used for amplification of factor SSEA-4 is shown in SEQ ID No.1, and a nucleotide sequence of a reverse primer sequence is shown in SEQ ID No.2;

A nucleotide sequence of a forward primer of a primer pair used for amplification of factor OCT-4 is shown in SEQ ID No.3, and a nucleotide sequence of a reverse primer sequence is shown in SEQ ID No.4;

A nucleotide sequence of a forward primer of a primer pair used for amplification of factor SOX-2 is shown in SEQ ID No.5, and a nucleotide sequence of a reverse primer sequence is shown in SEQ ID No.6;

A nucleotide sequence of a forward primer of a primer pair used for amplification of factor NANOG is shown in SEQ ID No.7, and a nucleotide sequence of a reverse primer sequence is shown in SEQ ID No.8;
3) Conducting electrophoresis detection on the amplification product obtained in step 2); when the amplified length of the amplification product is 121 bp, factor SSEA-4 is secreted beside hUC-MSCs;

When the amplified length of the amplification product is 137 bp, factor OCT-4 is secreted beside hUC-MSCs;

When the amplified length of the amplification product is 171 bp, factor SOX-2 is secreted beside hUC-MSCs;

When the amplified length of the amplification product is 94 bp, factor NANOG is secreted beside hUC-MSCs.

According to the present invention, RNA of hUC-MSCs is extracted and reversely transcribed into cDNA.

The present invention has no special restriction on extracting RNA of hUC-MSCs, and extraction can be conducted according to a kit. In the present invention, the system for the reverse transcription preferably comprises: 1 μL of dNTP (10 mM), 1 μL of Oligo dT-joint primer (2.5 mM), 1 μL of template RNA, 0.5 L of reverse transcriptase (500 U/μL), 0.5 μL of RNase inhibitor (40 U/μL), 4 μL of 5×RT-PCR buffer solution and 12 μL of double distilled water. In the present invention, the amplification procedure for the reverse transcription is preferably: replenishing the dNTP, the Oligo dT-joint primer and the template RNA to 10 μL with the double distilled water, pre-denaturing at 65° C. for 5 min, and mixing with the reverse transcriptase, the RNase inhibitor, the 5×RT-PCR buffer solution and the double distilled water; a reaction procedure is: 30° C. for 10 min, 42° C. for 15 min-30 min, 70° C. for 15 min, and 4° C. for preservation.

According to the present invention, PCR amplification is conducted on the cDNA obtained to obtain an amplification product; a nucleotide sequence of a forward primer of a primer pair used for amplification of factor SSEA-4 is shown in SEQ ID No.1, and a nucleotide sequence of a reverse primer sequence is shown in SEQ ID No.2; a nucleotide sequence of a forward primer of a primer pair used for amplification of factor OCT-4 is shown in SEQ ID No.3, and a nucleotide sequence of a reverse primer sequence is shown in SEQ ID No.4; a nucleotide sequence of a forward primer of a primer pair used for amplification of factor SOX-2 is shown in SEQ ID No.5, and a nucleotide sequence of a reverse primer sequence is shown in SEQ ID No.6; a nucleotide sequence of a forward primer of a primer pair used for amplification of factor NANOG is shown in SEQ ID No.7, and a nucleotide sequence of a reverse primer sequence is shown in SEQ ID No.8.

SEQ ID No. 1:
5′-atctacaacccagccttcttc-3′;
SEQ ID No. 2:
5′-cgttcacctcatcacacacat-3′;
SEQ ID No. 3:
5′-gaagcctttccccctgtctc-3′;
SEQ ID No. 4:
5′-aaaccctggcacaaactcca-3′;
SEQ ID No. 5:
5′-tcaggagttgtcaaggcagag-3′;
SEQ ID No. 6:
5′-cgccgccgatgattgttat-3′;
SEQ ID No. 7:
5′-caatggtgtgacgcagaagg-3′;
SEQ ID No. 8:
5′-aaggttcccagtcgggttca-3′.

In the present invention, the system for the PCR amplification preferably comprises: 2 μL of 10×PCR buffer solution, 0.8 μL of dNTP Mixture, 0.4 μL of forward primer (20 μM), 0.4 μL of reverse primer (20 μM), 0.2 μL of Ex Taq HS, 2 μL of cDNA and double distilled water replenished to 20 μL. In the present invention, the procedure for the PCR amplification is preferably: pre-denaturing at 94° C. for 5 min; denaturing at 94° C. for 30 s, annealing at 55° C. for 30 s, and extending at 72° C. for 30 s which are cycled for 35 times; extending at 72° C. for 10 min, and preserving at 4° C.

According to the present invention, electrophoresis detection is conducted on the amplification product obtained; when the amplified length of the amplification product is 121 bp, factor SSEA-4 is secreted beside hUC-MSCs; when the amplified length of the amplification product is 137 bp, factor OCT-4 is secreted beside hUC-MSCs; when the amplified length of the amplification product is 171 bp, factor SOX-2 is secreted beside hUC-MSCs; when the amplified length of the amplification product is 94 bp, factor NANOG is secreted beside hUC-MSCs. According to the present invention, a 2% agarose gel is prepared using an electrophoresis buffer solution (1×TBE or TAE) (EB is added at a temperature of about 55° C.-60° C. until the final concentration is 0.5 μg/mL. Staining may also be conducted after electrophoresis). 10 μL of the PCR amplification product is mixed with 2 μL of loading buffer solution, respectively, sample application is conducted, and 500 bp Ladder DNA Marker or a corresponding DNA marker is used to mark the relative molecular mass. Electrophoresis is conducted at a constant pressure of 3 V/cm-5 V/cm for 20 min. The gel is observed by an imager, recorded and analyzed.

To further describe the present invention, the prevent invention will be described below in detail in combination with embodiments, but the embodiments shall not be understood as the limitation of the protection scope of the present invention.

### Embodiment 1

### 1. Method and Principle

After DNA extraction of samples, primers are designed for mRNA sequences of the four multipotent genes of SSEA-4, OCT-4, SOX-2 and NANOG, cDNA samples are obtained by reverse transcription PCR, then PCR amplification is conducted using cDNA as a template, and the multipotency of hUC-MSCs at RNA level is qualitatively analyzed according to the amplification results. Reagents

All reagents are analytical reagents unless otherwise specified.

DEPC; absolute ethyl alcohol; glacial acetic acid; disodium ethylenediamine tetraacetic acid (Na₂EDTA·2H₂O); β-mercaptoethanol; trihydroxymethyl aminomethane (Tris); DNA molecular weight marker: (500 bp Ladder); ethidium bromide (EB) or other staining agents; a total RNA extraction kit: suitable for zooblasts; a reverse transcription kit; agarose (electrophoresis grade); DEPC water: 1 mL of DEPC is accurately transferred, added into 1000 mL of sterile ultra-pure water, and magnetically stirred at 37° C. overnight; 75% ethanol: after the DEPC water are subjected to autoclaved sterilization, 25 mL of DEPC water and 75 mL of absolute ethyl alcohol are mixed, and preserved at normal atmospheric temperature for later use; 50×TAE electrophoresis buffer solution: 242 g of Tris and 37.2 g of Na₂EDTA·2H₂O are accurately weighed and put into a 1 L beaker; about 800 mL of deionized water is added into the beaker and fully stirred for dissolving, 57.1 mL of acetic acid is added and fully stirred, and water is added to a volume of 1 L. When used, the electrophoresis buffer solution shall be diluted to 1×TAE;

Primers: primers for detecting the genes of SSEA-4, OCT-4, SOX-2 and NANOG as well as the internal control β-Actin and information thereof are shown in Table 1 below.

TABLE 1
Primers for multipotent genes of
SCLnow hUC-MSCs

| Gene to be | Primer sequence | Amplified |
|---|---|---|
| detected | (forward/reverse) | length |
| SSEA-4 | 5′-atctacaacccagccttcttc-3′ | 121 |
| | 5′-cgttcacctcatcacacacat-3′ | |
| OCT-4 | 5′-gaagcctttccccctgtctc-3′ | 137 |
| | 5′-aaaccctggcacaaactcca-3′ | |
| SOX-2 | 5′-tcaggagttgtcaaggcagag-3′ | 171 |
| | 5′-cgccgccgatgattgttat-3′ | |
| NANOG | 5′-caatggtgtgacgcagaagg-3′ | 94 |
| | 5′-aaggttcccagtcgggttca-3′ | |
| β-Actin | SEQ ID No.9: 5′-cctagaagcat | 432 |
| | ttgcggtgg-3′ | |
| | SEQ ID No.: 10: 5′- | |
| | gagctacgagctgcctgacgt-3′ | |

### 2. Materials

Centrifugal tubes, pipettes, tips, PCR reaction tubes (200 μL) and 96-well PCR reaction plates: sterile, non-pyrogenic and DNase/RNase free.

### 3. Instruments

A high-speed freezing centrifuge; a balance: with a reciprocal sensibility of 0.1 mg; an autoclave; a medical refrigerator: with a temperature range of-20° C.-4° C.; an ice machine; a microwave oven; a PCR instrument: 96-well gradient; electrophoresis apparatus: with a voltage of 10 V-300 V and a current of 4 mA-400 mA; a horizontal electrophoresis tank; micropipettes: with maximum ranges of 2.5 μL, 10 μL, 200 μL and 1000 μL, respectively; a gel imaging system.

### 4. Operating Procedure

### 4.1 Total RNA Extraction mRNA of hUC-MSCs is extracted according to a commercially available RNA extraction kit.

### 4.2 Synthesis of cDNA by Reverse Transcription

### 4.2.1 System for Reverse Transcription

The mRNA of SCLnow hUC-MSCs extracted is used as a template to conduct reverse

### transcription according to the instructions of a reverse transcription kit to obtain cDNA samples, and a system for reverse transcription is shown in Table 2.

TABLE 2
RT-PCR reaction system for multipotent
genes of SCLnow hUC-MSCs

| | | |
|---|---|---|
| | Stock solution | Volume of sample |
| Reagent name | concentration | in 20 μL system |

| | | | | |
|---|---|---|---|---|
| dNTP | 10 | mM | 1 | μL |
| Oligo dT-joint primer | 2.5 | μM | 1 | μL |
| Template RNA | 1-5 | μg | 1 | μL |
| Reverse transcriptase | 50 | U/μL | 0.5 | μL |
| RNase inhibitor | 40 | U/μL | 0.5 | μL |

| | | | |
|---|---|---|---|
| 5 × RT-PCR buffer | — | 4.0 | μL |
| solution | | | |
| (RNase free) Double | — | 12 | μL |
| distilled water | | | |

Note:
Amounts of reagents in the reaction system can be appropriately adjusted according to specific situations or different total volumes of reaction.
Note 2:
Two parallel reactions shall be arranged for each reaction system.

### 4.2.2 RT-PCR Parameters

The dNTP, the Oligo dT-joint primer and the template RNA are replenished to 10 μL with (RNase free) double distilled water, and pre-denatured at 65° C. for 5 min. The 10 μL of denatured mixture is replenished according to the system in the table, and a RT-PCR reaction procedure is: 30° C. for 10 min, 42° C. for 15 min-30 min, 70° C. for 15 min, and 4° C. for preservation.

### 4.3 Expression of Multipotent Genes Detected by PCR

### 4.3.1 PCR Reaction System

An RT-PCR reaction product is used as a template to conduct PCR amplification for multipotent genes of SCLnow hUC-MSCs, and a reaction system is shown in Table 3.

TABLE 3
PCR amplification reaction system for
multipotent genes of SCLnow hUC-MSCs

| | Stock solution | | Volume of sample | |
|---|---|---|---|---|
| Reagent name | concentration | | in 20 μL system | |
| 10 × PCR buffer solution | — | | 2 | μL |
| dNTP Mixture | 10 mM each | | 0.8 | μL |
| Upstream Primer | 20 | μM | 0.4 | μL |
| Downstream Primer | 20 | μM | 0.4 | μL |
| Ex Taq HS | 5 | U/μL | 0.2 | μL |

| | | | |
|---|---|---|---|
| cDNA (Reverse transcription | — | 2 | μL |

| | | |
|---|---|---|
| reaction solution) | | |
| (RNase free) Double | — | Up to 20 μL |
| distilled water | | |

Note 1:
Amounts of reagents in the reaction system can be appropriately adjusted according to specific situations or different total volumes of reaction.
Note 2:
Two parallel reactions shall be arranged for each reaction system.

### 4.3.2 Parameters of PCR Reaction Cycles

Pre-denaturing at 94° C. for 5 min. Denaturing at 94° C. for 30 s, annealing at 55° C. for 30 s, and extending at 72° C. for 30 s which are cycled for 35 times. Extending at 72° C. for 10 min, and preserving at 4° C.

Parameters of PCR reaction cycles may also be adjusted appropriately according to different gene amplification instruments.

### 5. Electrophoresis Detection of PCR Amplification Product

A 2% agarose gel is prepared using an electrophoresis buffer solution (1×TBE or TAE) (EB is added at a temperature of about 55° C.-60° C. until the final concentration is 0.5 μg/mL. Staining may also be conducted after electrophoresis). 10 μL of the PCR amplification product is mixed with 2 μL of loading buffer solution, respectively, sample application is conducted, and 500 bp Ladder DNA Marker or a corresponding DNA marker is used to mark the relative molecular mass. Electrophoresis is conducted at a constant pressure of 3 V/cm-5 V/cm for 20 min. The gel is observed by an imager, recorded and analyzed.

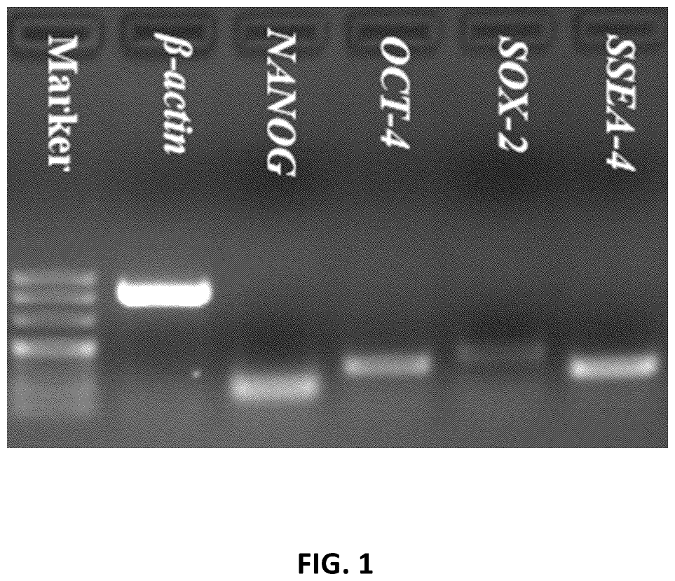

### 5.1 Result Determination (as Shown in FIG. 1)

Positive result: when the β-Actin is clear to the same degree in each lane, the multipotent genes of SCLnow hUC-MSCs show bright bands at a corresponding molecular weight;

Negative result: when the β-Actin is clear to the same degree in each lane, the multipotent genes of SCLnow hUC-MSCs do not show any band at a corresponding molecular weight.

The present invention is described in detail by the above embodiments. However, the above embodiments are merely part of the embodiments of the present invention, not all of the embodiments, and other embodiments can be obtained based on the embodiments in the present invention without contributing creative labor, which will also belong to the protection scope of the present invention.

What is claimed is:

1. A detection method for human umbilical cord mesenchymal stem cells (hUC-MSCs), comprising the following steps:
1) extracting RNA of hUC-MSCs and reversely transcribing the RNA into cDNA;
2) conducting PCR amplification on the cDNA obtained in step 1) to obtain an amplification product;
a nucleotide sequence of a forward primer of a primer pair used for amplification of factor SSEA-4 is shown in SEQ ID No.1, and a nucleotide sequence of a reverse primer sequence is shown in SEQ ID No.2;
a nucleotide sequence of a forward primer of a primer pair used for amplification of factor OCT-4 is shown in SEQ ID No.3, and a nucleotide sequence of a reverse primer sequence is shown in SEQ ID No.4;
a nucleotide sequence of a forward primer of a primer pair used for amplification of factor SOX-2 is shown in SEQ ID No.5, and a nucleotide sequence of a reverse primer sequence is shown in SEQ ID No.6;
a nucleotide sequence of a forward primer of a primer pair used for amplification of factor NANOG is shown in SEQ ID No.7, and a nucleotide sequence of a reverse primer sequence is shown in SEQ ID No.8;
3) conducting electrophoresis detection on the amplification product obtained in step 2); when the amplified length of the amplification product is 121 bp, factor SSEA-4 is secreted beside hUC-MSCs;
when the amplified length of the amplification product is 137 bp, factor OCT-4 is secreted beside hUC-MSCs;
when the amplified length of the amplification product is 171 bp, factor SOX-2 is secreted beside hUC-MSCs;
when the amplified length of the amplification product is 94 bp, factor NANOG is secreted beside hUC-MSCs.

2. The detection method according to claim 1, wherein a system for the reverse transcription in step 1) comprises: 1 μL of dNTP (10 mM), 1 μL of Oligo dT-joint primer (2.5 mM), 1 μL of template RNA, 0.5 μL of reverse transcriptase (500 U/μL), 0.5 L of RNase inhibitor (40 U/μL), 4 μL of 5×RT-PCR buffer solution and 12 μL of double distilled water.

3. The detection method according to claim 2, wherein an amplification procedure for the reverse transcription is: replenishing the dNTP, the Oligo dT-joint primer and the template RNA to 10 UL with the double distilled water, pre-denaturing at 65° C. for 5 min, and mixing with the reverse transcriptase, the RNase inhibitor, the 5×RT-PCR buffer solution and the double distilled water; a reaction procedure is: 30° C. for 10 min, 42° C. for 15 min-30 min, 70° C. for 15 min, and 4° C. for preservation.

4. The detection method according to claim 1, wherein a system for the PCR amplification in step 2) comprises: 2 μL of 10×PCR buffer solution, 0.8 μL of dNTP Mixture, 0.4 μL of forward primer (20 μM), 0.4 μL of reverse primer (20 μM), 0.2 μL of Ex Taq HS, 2 μL of cDNA and double distilled water replenished to 20 μL.

5. The detection method according to claim 1, wherein a procedure for the PCR amplification is: pre-denaturing at 94° C. for 5 min; denaturing at 94° C. for 30 s, annealing at 55° C. for 30 s, and extending at 72° C. for 30 s which are cycled for 35 times; extending at 72° C. for 10 min, and preserving at 4° C.


SEQUENCE LISTING (10 sequences)

SEQ ID NO: 1 - DNA, 21 bp, unidentified
  source 1..21: mol_type = other DNA
atctacaacccagccttcttc

SEQ ID NO: 2 - DNA, 21 bp, unidentified
  source 1..21: mol_type = genomic DNA
cgttcacctcatcacacacat

SEQ ID NO: 3 - DNA, 20 bp, unidentified
  source 1..20: mol_type = genomic DNA
gaagcctttccccctgtctc

SEQ ID NO: 4 - DNA, 20 bp, unidentified
  source 1..20: mol_type = genomic DNA
aaaccctggcacaaactcca

SEQ ID NO: 5 - DNA, 21 bp, unidentified
  source 1..21: mol_type = other DNA
tcaggagttgtcaaggcagag

SEQ ID NO: 6 - DNA, 19 bp, unidentified
  source 1..19: mol_type = genomic DNA
cgccgccgatgattgttat

SEQ ID NO: 7 - DNA, 20 bp, unidentified
  source 1..20: mol_type = genomic DNA
caatggtgtgacgcagaagg

SEQ ID NO: 8 - DNA, 20 bp, unidentified
  source 1..20: mol_type = genomic DNA
aaggttcccagtcgggttca

SEQ ID NO: 9 - DNA, 20 bp, unidentified
  source 1..20: mol_type = other DNA
cctagaagcatttgcggtgg

SEQ ID NO: 10 - DNA, 21 bp, unidentified
  source 1..21: mol_type = genomic DNA
gagctacgagctgcctgacgt